(19) United States
(12) Patent Application Publication
(10) Pub. No.: US 2026/0102558 A1
(43) Pub. Date: Apr. 16, 2026

(54) System for Auto-Administration of Medication

(71) Applicants: Sarah Vohra, Cupertino, CA (US); Ronav Mehul Dholakia, Cupertino, CA (US)

(72) Inventors: Sarah Vohra, Cupertino, CA (US); Ronav Mehul Dholakia, Cupertino, CA (US)

(21) Appl. No.: 19/354,572

(22) Filed: Oct. 9, 2025

Related U.S. Application Data
(60) Provisional application No. 63/705,921, filed on Oct. 10, 2024.

(51) Int. Cl. A61M 5/142; A61M 5/145; A61M 5/31; A61M 5/315

(52) U.S. Cl. CPC A61M 5/14216; A61M 5/1452; A61M 5/3129; A61M 5/3148; A61M 5/31511; A61M 5/31578; A61M 2005/14208; A61M 2202/0007; A61M 2202/0468; A61M 2205/103; A61M 2205/18; A61M 2205/3303; A61M 2205/3327; A61M 2205/3553; A61M 2205/3576; A61M 2205/50; A61M 2205/8206; A61M 2230/005; A61M 2230/40

(57) ABSTRACT

The system for auto-administration of medication provides an antidote to opioid overdoses based on respiration rates and/or blood oxygen level (also known as oxygen saturation level). The system and method for auto-administration of medication seeks to provide users with a device that utilizes biosensors to detect respiratory depression in a user. The system monitors the user's respiratory rate and/or blood oxygen level and sends communication signals to a physical apparatus that then administers an antidote for opioid overdoses once a signal is received through the signaling module.

### FIELD OF THE INVENTION

The present invention relates generally to a system and method that administers a medication. More specifically, the present invention is a device that administers Naloxone to a user who is suspected of having an opioid overdose.

### BACKGROUND OF THE INVENTION

Opioid overdoses are a critical public health crisis, wherein opioids such as heroin, prescription painkillers, and synthetic opioids like fentanyl are contributing and causing a large number of fatalities. During an opioid overdose, the opioid suppresses the respiratory system, leading to dangerously slow or stopped breathing. Opioid overdose symptoms include unconsciousness, slow or absent breathing, and blue or cold skin.

Naloxone, commonly known by its brand name Narcan, is a life-saving medication that can reverse the effects of an opioid overdose if administered promptly. It works by rapidly binding to the opioid receptors in the brain, displacing the opioids that are causing the overdose, and temporarily blocking their effects. Naloxone can be administered through injection or via a nasal spray, making it relatively easy for first responders, bystanders, or the individual at risk to administer during an emergency. Due to opioid overdoses often occurring quickly, the accessibility of Naloxone has become a key factor in preventing overdose deaths. Many states have expanded access to naloxone by allowing it to be sold over-the-counter without a prescription, and by training law enforcement, emergency medical personnel, and even the general public on its use.

An objective of the present invention is to provide users with a device that detects an opioid overdose in an individual and promptly administers Naloxone. The present invention intends to provide users with a device that prevents death from an overdose if the individual overdosing is alone. In order to accomplish that, a preferred embodiment of the present invention comprises a monitor, a signaling module, and a physical apparatus. Thus, the present invention is a system that detects an opioid overdose in an individual and provides an auto administration of Naloxone to prevent an opioid overdose death.

### BRIEF DESCRIPTION OF THE DRAWINGS

FIG. 1 is a block diagram of the present invention.

FIG. 2 is a flow diagram view of the present invention.

FIG. 3 is a perspective view of the present invention.

FIG. 4 is a front view of the present invention.

FIG. 5 is a cross-sectional view of the present invention.

### DETAILED DESCRIPTION OF THE INVENTION

All illustrations of the drawings are for the purpose of describing selected versions of the present invention and are not intended to limit the scope of the present invention.

The present invention is a system that provides an antidote to opioid overdoses based on respiration rates and/or blood oxygen level (also known as oxygen saturation level). The present invention seeks to provide users with a device that utilizes a biosensor 12 to detect respiratory depression in a user. In order to accomplish this the present invention comprises a monitor 1 that measures the user's respiratory rate and/or blood oxygen level and sends communication signals. Further, the signaling module 2 translates the data from the monitor 1 into instructive signals for the physical apparatus 3. Additionally, the physical apparatus 3 administers an antidote for opioid overdoses once a signal is received through the signaling module 2. Thus, the present invention is a system that detects an opioid overdose in an individual and provides an auto administration of Naloxone to prevent an opioid overdose death.

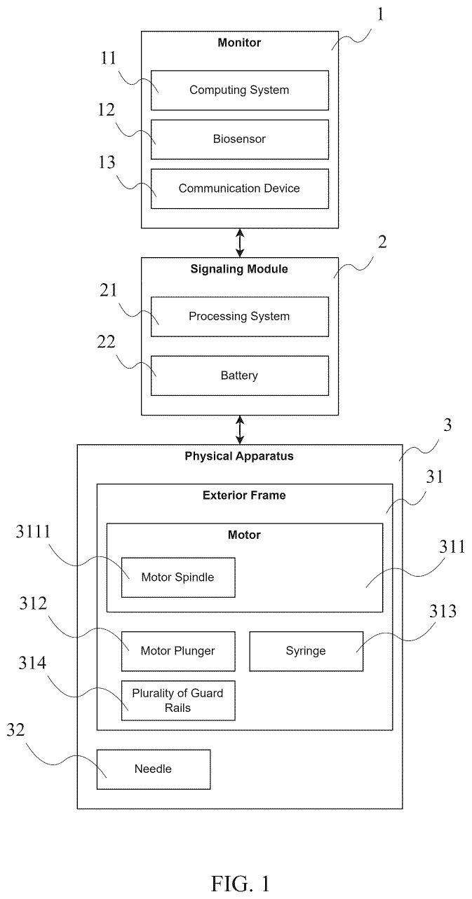

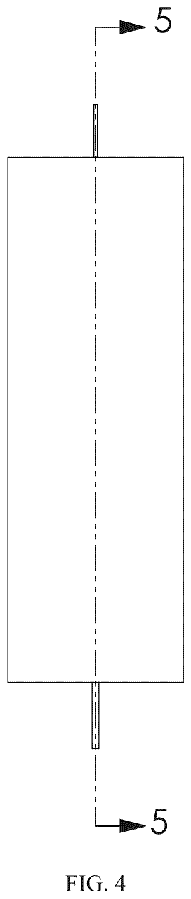

As shown in FIGS. 1-5, the present invention is a device that eliminates the need for a bystander when an individual is experiencing an opioid overdose. An objective of the present invention is to provide users with a system that administers an opioid overdose antidote to prevent deaths from opioid overdoses. The present invention intends to provide users with a device that detects respiratory depression in a user. Respiratory depression is a decreased respiratory rate of less than 8 breaths per minute or decreased blood oxygen level (O2 saturation less than 90%) sustained over a period of 3 minutes, in the setting of opioid use. To accomplish this the present invention comprises a monitor 1, a signaling module 2, and a physical apparatus 3. The signaling module 2 remotely communicates with the monitor 1. Thus, the present invention is a system that detects an opioid overdose in an individual and provides an auto administration of a medication to prevent an opioid overdose death.

The present invention comprises a monitor 1, a physical apparatus 3, and a signaling module 2. The monitor 1 measures the respiratory rate and/or blood oxygen level of a user. The monitor 1 comprises a computing system 11, a biosensor 12, and a communication device 13. The biosensor 12 is wearable and supports Android or iOS APIs or other operating systems. The computing system 11 operates a software application that obtains health metrics such as a user's respiratory rate and/or blood oxygen level to then trigger the release of a drug. The software application utilizes various algorithms to detect symptoms of an opioid overdose wherein the user has a decreased respiratory rate of less than eight breaths per minutes or decreased blood oxygen level (oxygen saturation less than 90%) sustained over a period of three minutes. The communication device 13 utilizes an appropriate means to communicate with the signaling module 2. In this embodiment, it utilizes Bluetooth technology to communicate with the signaling module 2. The communication device 13 further utilizes cell tower connectivity to send alerts and make calls to emergency operators. The signaling module 2 enables a detected health metric to control the output of a medication such as Naloxone. The signaling module 2 comprises a processing system 21, and a battery 22. The processing system 21 translates the signal received from the monitor 1 into a control command for the physical apparatus 3. Thus, the signaling module 2 directly controls the physical apparatus 3 based on the signals received from the monitor 1. The battery 22 is rechargeable and further provides electrical power for both the signaling module 2 and the physical apparatus 3. The physical apparatus 3 receives signals from the signaling module 2 to which the physical apparatus 3 is electrically coupled. The physical apparatus 3 is designed to deliver a drug to the user without the need for a bystander. The physical apparatus 3 comprises a housing 31 and a needle 32. The housing 31 is attachable to the user and the needle 32 is insertable into the user. The monitor 1 is remotely coupled to the signaling module 2, to enable the monitor 1 to send signals remotely to the signaling module 2 without a physical connection. The signaling module 2 is electronically coupled to the physical apparatus 3. Thus, the signaling module 2 directly controls the physical apparatus 3 based on the signal received from the monitor 1.

The monitor 1 is a wearable device that monitors biomarkers from the body of the user. The monitor 1 is configured to detect respiratory depression in a user. As shown in FIG. 1, the computing system 11, biosensor 12 and communication device 13 are electronically coupled to one another. This enables the computing system 11 to analyze data received from the biosensor 12 and send said data as a signal through the communication device 13.

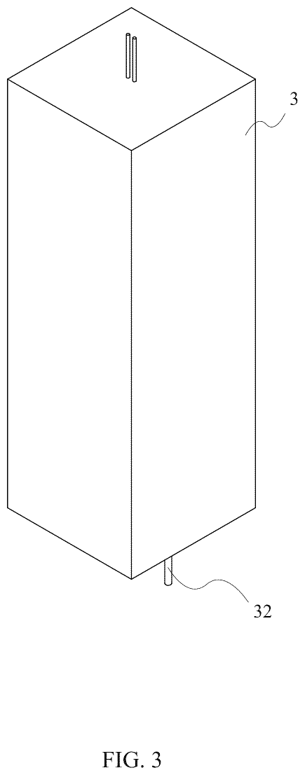

In reference to FIG. 3, the needle 32 is integrated along the bottom of the housing 31. The needle 32 is injected into the user, to ensure the medication may be supplied to the user as needed. The housing 31 comprises a motor 311, a motor plunger 312, a syringe 313, and a plurality of guide rails 314. The motor 311 further comprises a motor spindle 3111. The motor spindle 3111 traverses through the mechanical assembly. The motor plunger 312 is mechanically coupled to the motor spindle 3111 wherein the motor plunger 312 actuates upwards and downwards as the motor spindle 3111 rotates. Thus, the motor plunger 312 is moved and controlled by the rotation of the motor 311 and the attached motor spindle 3111. The motor plunger 312 is connected to the top of the syringe 313. The syringe 313 may expand and contract in length in tandem with the movement of the motor plunger 312. As a portion of the syringe 313 actuates downwards and contracts in length, whatever is housed within the syringe 313 is pushed out through the needle 32. The motor spindle 3111 is a threaded rod that extends downwards from the motor 311 within the housing 31. The motor spindle 3111 rotates along the central axis of the motor spindle 3111, within the motor 311. The motor 311 is electrically coupled to the signaling module 2.

The syringe 313 houses a medication. The preferred medication is Naloxone wherein up to two, four mg doses of Naloxone can be administered to the user in the event that the monitor 1 detects an opioid overdose. The syringe 313 is fluidly coupled to the needle 32, wherein the medication may flow from the syringe 313 into the needle 32 and into the user. The needle 32 is always subcutaneously inserted in the user.

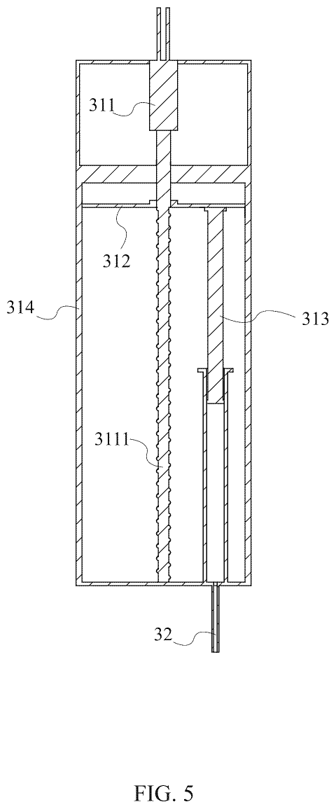

In reference to FIG. 5, the plurality of guide rails 314 is integrated within the housing 31. The plurality of guide rails 314 traverses the height of the mechanical assembly inner surface. The plurality of guide rails 314 engages with the motor plunger 312 to keep the motor plunger 312 aligned. As a result, the motor plunger 312 stays aligned as the motor plunger 312 moves upwards and downwards within the mechanical assembly.

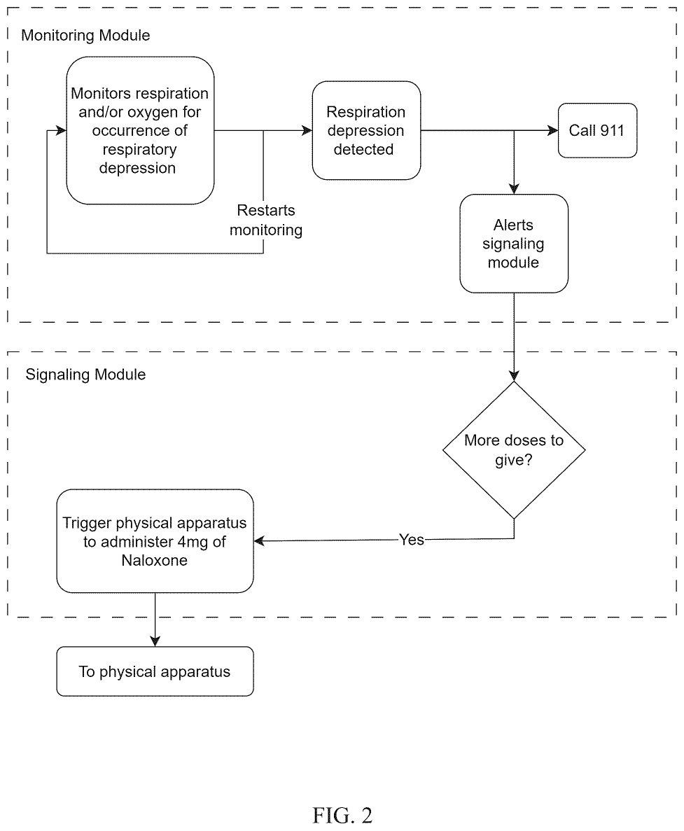

In reference to FIG. 2, the method for administering a medication to a user begins with the monitor 1 detecting respiratory depression in the user. Respiratory depression is a decreased respiratory rate of less than 8 breaths per minute or decreased blood oxygen level (O2 saturation less than 90%) sustained over a period of 3 minutes, in the setting of opioid use. The communication device 13 calls an emergency service. The communication device 13 sends an alert to the signaling module 2. The signaling module 2 communicates with the monitor 1 via Bluetooth and communicates with the physical apparatus 3 via an Arduino Nano BLE in this embodiment. In an alternative embodiment, communication means could use wired or wireless technologies in place of Bluetooth or other processing units in place of the Arduino Nano BLE. The signaling module 2 processes the alert. The signaling module 2 activates the motor 311. The physical apparatus 3 administers a dose of the medication to the user. In the preferred embodiment the physical apparatus 3 administers 4 mg of Naloxone to the user.

The process repeats under certain conditions as seen in FIG. 2, the monitor 1 continues to check the respiration rate of the user for three minutes, after first detecting respiratory depression and alerting the signaling module. The Arduino Nano BLE utilizes a software wherein a signal is sent to the physical apparatus 3 for a pre-calibrated length of time to ensure the physical apparatus 3 delivers the correct dosage of the desired drug to the user. The signaling module 2 sends a signal to the physical apparatus 3 any time the monitor 1 detects respiratory depression by detecting a drop in respiratory rate and/or blood oxygen level for a sustained 3 minute period, and the number of doses administered is under the allowable limit. In this embodiment, the limit is 2 doses of 4 mg each of Naloxone. The monitor 1 checks blood oxygen level of the user for three minutes, after first detecting respiratory depression. If the monitor 1 detects respiratory depression, then the monitor 1 sends another alert to the signaling module 2. The process repeats until the monitor 1 does not detect respiratory depression. If the monitor 1 does not detect respiratory depression then the monitor 1 does not send an alert to the signaling module 2.

Although the invention has been explained in relation to its preferred embodiment, it is to be understood that many other possible modifications and variations can be made without departing from the spirit and scope of the invention as hereinafter claimed.

What is claimed is:

1. A system for auto-administration of medication comprising:
a monitor, a physical apparatus, and a signaling module;
the monitor comprising a computing system, a biosensor, and a communication device;
the signaling module comprising a processing system, and a battery;
the physical apparatus comprising a housing and a needle;
the monitor is remotely coupled to the signaling module; and
the signaling module is electronically coupled to the physical apparatus.

2. The system for auto-administration of medication as claimed in claim 1 wherein the monitor is a wearable device that monitors biomarkers from the body of the user.

3. The system for auto-administration of medication as claimed in claim 1 wherein the computing system, biosensor and communication device are electronically coupled to one another.

4. The system for auto-administration of medication as claimed in claim 1 comprising:
the needle is integrated along the bottom of the housing;
the housing comprising a motor, a motor plunger, a syringe, and a plurality of guide rails; and
the motor further comprising a motor spindle.

5. The system for auto-administration of medication as claimed in claim 4 wherein the needle is injected into the user.

6. The system for auto-administration of medication as claimed in claim 4 comprising:
the motor plunger is mechanically coupled to the motor spindle wherein the motor plunger actuates upwards and downwards as the motor spindle rotates;
the motor plunger is connected to the top of the syringe; and
the syringe may expand and contract in length in tandem with the movement of the motor plunger.

7. The system for auto-administration of medication as claimed in claim 6 comprising:
the motor spindle is a threaded rod that extends downwards from the motor within the housing;
the motor spindle rotates along the central axis of the motor spindle, within the motor; and
the motor is electrically coupled to the signaling module.

8. The system for auto-administration of medication as claimed in claim 4 wherein the syringe houses a medication.

9. The system for auto-administration of medication as claimed in claim 8 wherein the syringe being fluidly coupled to the needle, wherein the medication may flow from the syringe into the needle and into the user.

10. The system for auto-administration of medication as claimed in claim 4 comprising:
the plurality of guide rails is integrated within the housing; and
the plurality of guide rails engages with the motor plunger to keep the motor plunger aligned.

11. A method for auto-administration of medication comprising:
a monitor detects respiratory depression in a user;
a communication device calls an emergency service;
the communication device sends alert to a signaling module;
the signaling module processes the alert;
the signaling module activates a motor within a physical apparatus; and
a physical apparatus administers a dose of a medication to the user.

12. The method for auto-administration of medication as claimed in claim 11 comprising:
the monitor checks respiration rate of the user for three minutes, after first detecting respiratory depression; and
the monitor checks blood oxygen level of the user for three minutes, after first detecting respiratory depression.

13. The method for auto-administration of medication as claimed in claim 12 comprising:
if the monitor detects respiratory depression; and
the monitor sends another alert to the signaling module.

14. The method for auto-administration of medication as claimed in claim 13 wherein the process repeats until the monitor does not detect respiratory depression.

15. The method for auto-administration of medication as claimed in claim 12 comprising:
if the monitor does not detect respiratory depression; and
the monitor does not send an alert to the signaling module.

16. The method for auto-administration of medication as claimed in claim 11 comprising:
the physical apparatus administers 4 mg of Naloxone to the user; and
wherein a maximum of two 4 mg doses is administered to the user.

17. A system for auto-administration of medication comprising:
a monitor, a physical apparatus, and a signaling module;
the monitor comprising a computing system, a biosensor, and a communication device;
the signaling module comprising a processing system, and a battery;
the physical apparatus comprising a housing and a needle;
the monitor is remotely coupled to the signaling module;
the signaling module is electronically coupled to the physical apparatus;
wherein the monitor is a wearable device that monitors biomarkers from the body of the user;
the computing system, biosensor and communication device are electronically coupled to one another;
the needle is integrated along the bottom of the housing;
the housing comprising a motor, a motor plunger, a syringe, and a plurality of guide rails;
the motor further comprising a motor spindle;
the motor plunger is mechanically coupled to the motor spindle wherein the motor plunger actuates upwards and downwards as the motor spindle rotates; and
wherein the syringe houses a medication.

18. The system for auto-administration of medication as claimed in claim 17 wherein the needle is injected into the user.

19. The system for auto-administration of medication as claimed in claim 17 comprising:
the motor plunger is connected to the top of the syringe;
the syringe may expand and contract in length in tandem with the movement of the motor plunger;
the motor spindle is a threaded rod that extends downwards from the motor within the housing;
the motor spindle rotates along the central axis of the motor spindle, within the motor;
the motor is electrically coupled to the signaling module; and
the syringe being fluidly coupled to the needle, wherein the medication may flow from the syringe into the needle and into the user.

20. The system for auto-administration of medication as claimed in claim 17 comprising:
the plurality of guide rails is integrated within the housing; and
the plurality of guide rails engages with the motor plunger to keep the motor plunger aligned.
